(19) United States
(12) Patent Application Publication
(10) Pub. No.: US 2014/0171920 A1
(43) Pub. Date: Jun. 19, 2014

(54) WOUND DRESSING APPARATUSES AND METHODS THEREOF

(71) Applicants: Christopher Scott Smith, Suffolk, VA (US); Christopher Hugh Freisenbruch, Virginia Beach, VA (US)

(72) Inventors: Christopher Scott Smith, Suffolk, VA (US); Christopher Hugh Freisenbruch, Virginia Beach, VA (US)

(21) Appl. No.: 14/107,809

(22) Filed: Dec. 16, 2013

Related U.S. Application Data
(60) Provisional application No. 61/737,743, filed on Dec. 15, 2012.

(51) Int. Cl. A61F 13/02; A61M 1/00

(52) U.S. Cl. CPC A61F 13/0216; A61M 1/0066
USPC 604/540

(57) ABSTRACT

A wound dressing apparatus includes a patient interface configured to attach to an area of skin on a patient and extend around a wound on the patient. An access port extends from the patient interface and includes an aperture in the patient interface configured to provide access to the wound. An attachment structure extends about and out from an outer boundary of the access port. An access cap is configured to be removably attached to the attachment structure to provide an occlusive seal over the wound when attached to the attachment structure. A method of treatment utilizing the wound dressing apparatus includes attaching the patient interface to the area of skin on the patient extending around the wound of the patient. The access cap is removed from the attachment structure to relieve pressure from the wound. A treatment device is attached to the attachment structure of the wound dressing apparatus.

This application claims the benefit of U.S. Provisional Application No. 61/737,743, filed Dec. 15, 2012 which is hereby incorporated by reference in its entirety.

### FIELD

This technology generally relates to a wound dressing apparatus and, more particularly, to wound dressing apparatuses which may include an access port and a removable cap that allow for the attachment of various treatment devices and methods of use thereof.

### BACKGROUND

Airway, Breathing and Circulation or ABCs are the basics tenets for treatment of a patient in emergency situations. Time, training, space, and environment are all limiting factors of what can be done by a medic in a field environment. Each of these limiting factors work against a patient's chance for survival. By mitigating these limiting factors, the chances for saving the patient significantly increase.

For example, the simpler the treatments are the less training is required and the quicker the treatment can be applied in a stressful field setting. The more versatile the tools in the medic's aid bag, the more space is available in the aid bag to bring additional equipment. Additionally, medical tools that require a lesser amount of manipulation by the medic once they are in use will have less of a chance of eventually failing. Further, tools that permit the medic to visually reassess the effectiveness of the treatment are desired. The availability of medical devices that can be easily manipulated manually and that do not require electricity to function allows the medic to apply a higher echelon of care in the field.

In a field setting, the standard treatment for a chest wound is to seal off the wound with a chest seal to prevent air from entering the chest cavity. Application of the chest seal results in a build up of pressure in the chest cavity. The medic must relieve the pressure created by the chest seal to prevent further collapse of the patient's lungs.

Current chest seals utilize either an occlusive dressing or a one way valve dressing. Occlusive chest seals, by nature, trap air within the wound causing the pressure to build. Such seals require the medic to relieve the pressure by lifting the seal. This technique is commonly referred to as “burping” the wound. Chest seals with one way valves allow air to escape from the wound site to eliminate the need to burp the wound. Such chest seals, however, are susceptible to occlusion by debris or blood from the wound site. After occlusion of the one way valve, the medic is forced to burp the wound by lifting the seal. In either case, burping the wound results in a loss of adhesion for the chest seal, which reduces effectiveness or forces the medic to change to a new dressing, which increases the number of supplies utilized.

Treatment with a chest seal alone also does not provide any treatment for lost lung function resulting from a chest wound. The best a medic can hope for is to relieve the pressure to prevent further damage to the collapsed lung. Thus, treatment of the chest compromise after applying the initial dressing is desirable. The definitive treatment for chest compromise in a non-ideal field setting is the eventual placement of a chest tube. For example, the standard for all Special Forces Medics is to be able to care for patients in a field setting for up to 72 hours. Therefore, the ability to apply a chest tube to a wounded patient in extended scenarios is a must for the patient's survival. However, when applying a chest tube in a non-ideal field setting, a substantial issue arises as to how suction can be applied once the tube is in place without the benefit of electricity.

### SUMMARY

A wound dressing apparatus includes a detachable patient interface configured to attach an area of skin on a patient and extend around a wound on the patient. An access port extends from the patient interface and includes an aperture in the patient interface that provides access to the wound. An attachment structure extends about and out from an outer boundary of the access port. An access cap is configured to be removably attached to the attachment structure to provide an occlusive seal over the wound when attached to the attachment structure. The wound dressing apparatus may further include a treatment device capable of being operably attached to the attachment structure after the access cap is removed.

A method of treating a wound on a patient includes providing a wound dressing apparatus. The wound dress apparatus includes a detachable patient interface configured to attach to an area of skin on the patient and extend around the wound on the patient. An access port extends from the patient interface and includes an aperture in the wound dressing apparatus configured to provide access to the wound. An attachment structure extends about and out from an outer boundary of the access port. An access cap is configured to be removably attached to the attachment structure to provide an occlusive seal over the wound when attached to the attachment structure. The patient interface is attached to the area of skin on the patient extending around the wound of the patient. The access cap is removed from the attachment structure to relieve pressure from the wound. The method may further include providing a treatment device capable of being operably attached to the attachment structure after the access cap is removed. The treatment device is attached to the attachment structure of the wound dressing apparatus.

This exemplary technology provides a number of advantages including providing more effective and efficient wound dressing apparatuses and methods of use thereof. For example, this technology provides a wound dressing that permits access to the wound without comprising the adhesive attachment of the dressing to the patient. Additionally, this technology provides a wound dressing that interchangeably accepts various treatment devices that may advantageously be utilized in the field without the need for a high level of medical training.

The wound dressing apparatus advantageously includes a wound access port with a removable access cap. The access cap, when attached, provides an occlusive dressing that seals and protects the wound. Removal of the access cap facilitates the release of pressure from the wound site while maintaining the adhesive attachment of the dressing around the wound site. Removal of the access cap also facilitates the attachment of various treatment options that can be used where pump-enabled suction is required and electricity is not available.

The wound dressing apparatus is easily portable and can be carried in a field medic's aid bag. This is a significant departure from existing field-applicable medical devices which are not portable and/or are too bulky or heavy to be carried in the medic's aid bag. The wound dressing apparatus design provides additional and advanced treatment options that are not available with existing field medical devices. In particular, the universal attachment section of the wound dressing apparatus allows a field medic to reuse one or more components with different attachments to perform different medical treatments as the medic progresses with the overall treatment on the patient.

The wound dressing apparatus is versatile enough to reduce the need for multiple pieces of bulky equipment by being configured to universally adapt to multiple different medical tools that are commonly found in a field medic's aid-bag. The versatility of the wound dressing apparatus therefore expands treatment protocols, allowing the medic to achieve more with less. The universal attachment section employed in the wound dressing apparatus allows effective coupling and connection of various treatment devices with any commonly used insertion tube, to for example, effectively provide suction. In an aspect, the wound dressing apparatus can also be used as a field expedient bag valve mask.

An additional advantage of the wound dressing apparatus and the associated treatment devices is that they do not require electricity or an external power source to operate, but instead operate using manual manipulation by a person and/or leveraging gravity. This makes the wound dressing apparatus and associated treatment devices ideal in a field setting, such as a combat theatre, where a power source may not be available.

Another advantage of wound dressing apparatus and associated treatment devices is that they are designed to be easily used by either an experienced or novice field medic to conduct advanced treatments in the field. For example, the wound dressing apparatus and associated treatment devices are designed to be easily used in an advanced treatment like relieving pressure on the lungs and heart through an existing wound, thereby reducing or eliminating pain or more injury to the patient by needle decompression or emplacement of a chest tube.

### BRIEF DESCRIPTION OF THE DRAWINGS

FIG. 1A is an exploded side view of an example of a wound dressing apparatus and associated access cap;

FIG. 1B is a top view of an access port and attachment section of the wound dressing apparatus of FIG. 1A;

FIGS. 1C and 1D are a top and bottom view of an access cap of the wound dressing apparatus;

FIG. 2A is a side view of an example of a dual pump device capable of being attached to the wound dressing apparatus;

FIGS. 2B and 2C are side views of the dual pump device of FIG. 2A in operable attachment to the wound dressing apparatus;

FIG. 3A is a side of an example of a thoracic catheter device capable of being attached to the wound dressing apparatus;

FIG. 3B is a side view of the thoracic catheter device of FIG. 3A in operable attachment to the wound dressing apparatus;

FIG. 4A is a side view of an example of the wound dressing apparatus with a thoracic trocar device capable of being attached to the wound dressing apparatus;

FIG. 4B is an exploded side view of the thoracic trocar device of FIG. 4A;

FIG. 4C is a side view of the thoracic trocar device of FIG. 4A in operable attachment to the wound dressing apparatus; and

FIGS. 4D-4F are isometric views of the three way stop cock of thoracic trocar device of FIG. 4A in various positions.

### DETAILED DESCRIPTION

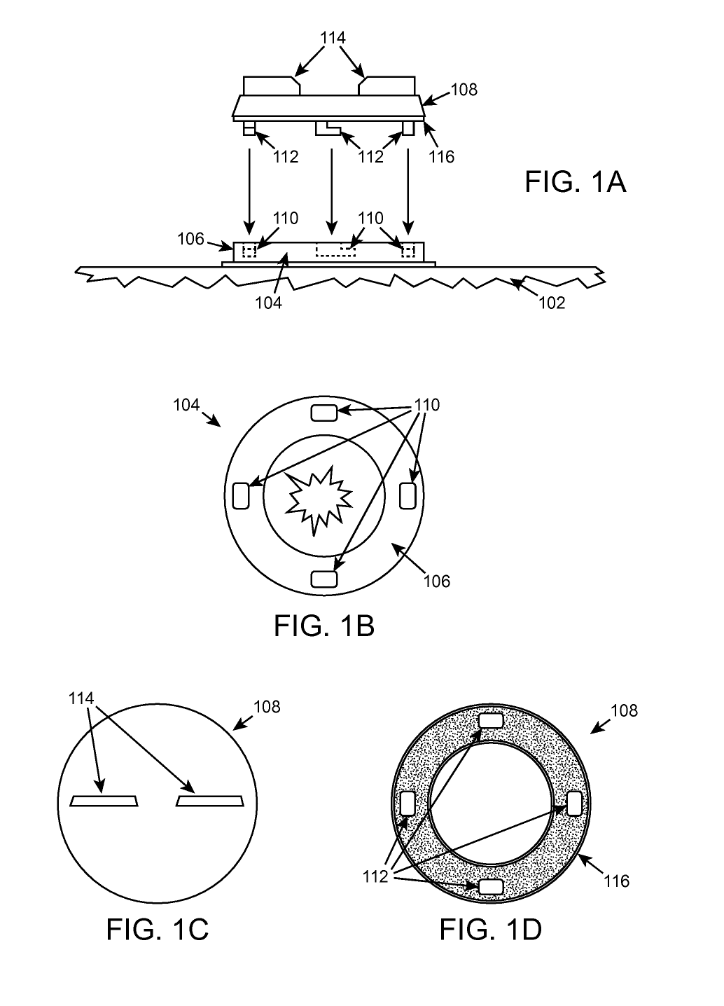

An exemplary wound dressing apparatus 100 is illustrated in FIGS. 1A-1D. The exemplary wound dressing apparatus 100 includes a patient interface 102, an access port 104, an attachment section 106, and a removable access cap 108, although the wound dressing apparatus 100 could include other type and numbers of components and other elements in other configurations. This exemplary technology provides a number of advantages including providing an efficient and cost-saving wound dressing that permits access to a wound site after dressing without removal of the entire wound dressing apparatus. The technology also provides the ability to attach various treatment devices to the wound dressing apparatus.

Referring more specifically to FIG. 1A, the patient interface 102 is configured to attach to an area of skin on a patient and extend around a wound on the patient, although the patient interface 102 may have other configurations. The patient interface 102 may be constructed from and have the configuration of any wound dressing material known in the art, except as described below. The patient interface 102 includes an adhesive layer, such as a dehydrated Hydrogel adhesive, to detachably attach the patient interface 102 to the skin of the patient, although other skin attachment methods may be utilized.

Referring now to FIGS. 1A and 1B, access port 104 extends from the patient interface 102 and includes an aperture defined within the access port 104. The access port 104 is configured to provide access to a patient's wound or incision site once the patient interface 102 is applied to the skin of the patient. In one example, the access port 104 includes a circular aperture concentric with the access port 104, although other configurations suitable to extend around a wound site on a patient may be contemplated.

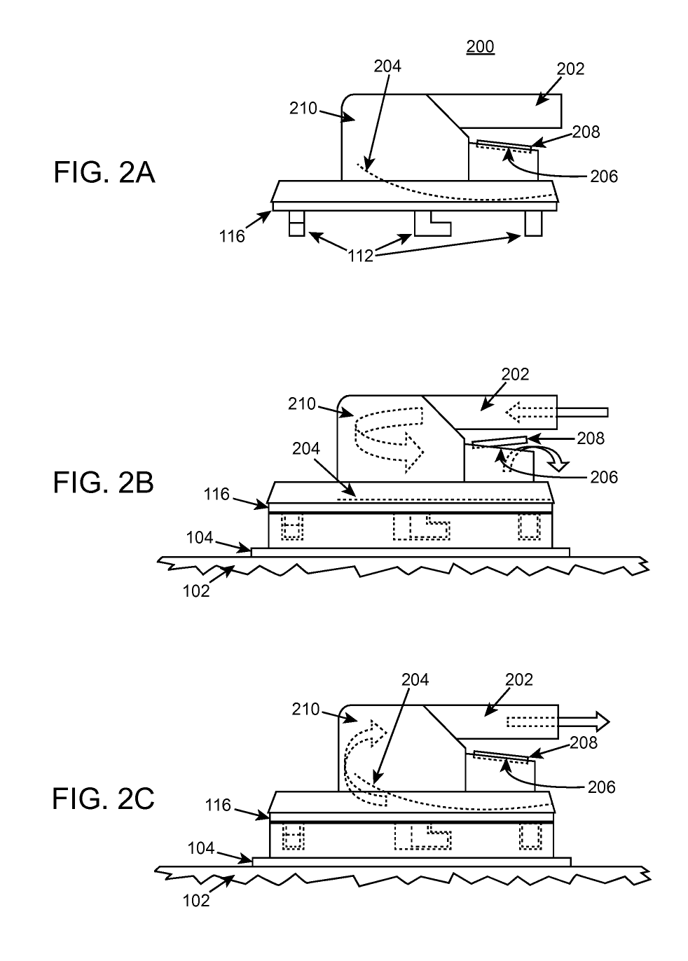

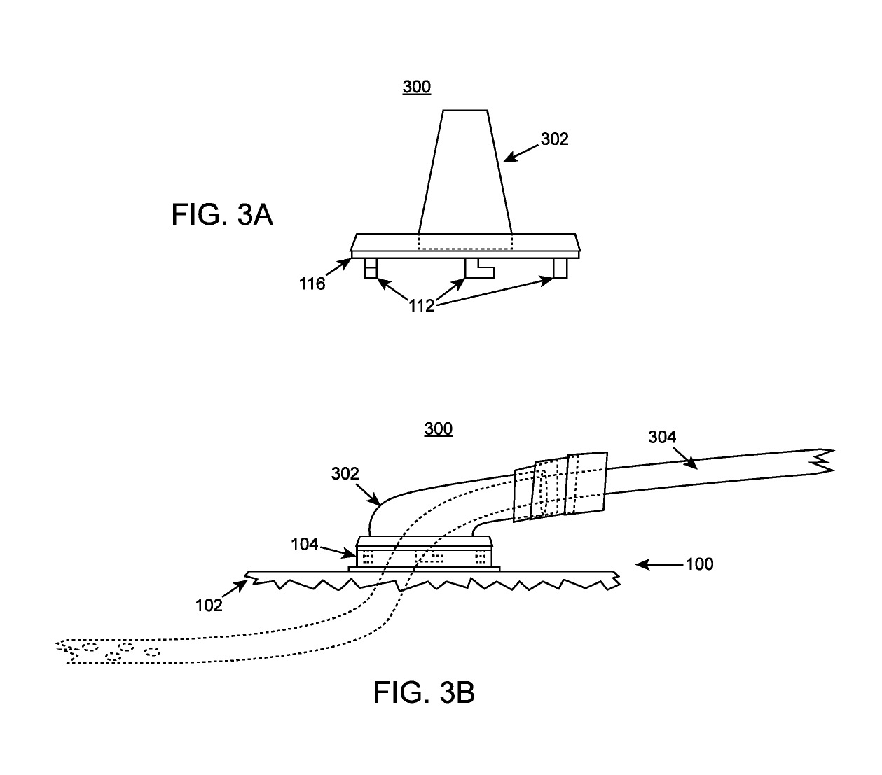

Attachment structure 106 extends about and out from an outer boundary of the access port 104, although attachment structure 106 may have other configurations in other locations on the wound dressing apparatus 100. The attachment structure 106 is configured to receive and provide removable attachment of various adaptors, including by way of example access cap 108, although attachment structure 106 may receive and provide removable attachment of other various treatment devices or adaptors as illustrated, by way of example only, in FIGS. 2A, 3A, and 3B, which are described in detail below. Attachment structure 106 may include one or more attachment mechanisms configured to mate access cap 108 to attachment structure 106, such as, by way of example only, receiving slots 110. Receiving slots 110 are adapted to receive a portion of the access cap 108 and mechanically secure the access cap 108 to attachment structure 106. Although the use of receiving wells 110 is described, attachment section 106 may include any other attachment mechanisms, such as by way of example only, magnets, tabs, mating threads, or adhesives, in order to removably attach the access cap 108 to the attachment section 106.

Referring now to FIGS. 1A, 1C, and 1D, access cap 108 is configured to be removably attached to the attachment structure 106 to cover access port 104. The access cap 108 provides an occlusive seal over the wound when attached to the attachment structure 106. Access cap 108 includes one or more attachment mechanisms configured to mate access cap 108 to attachment structure 106, such as, by way of example only, tabs 112. Tabs 112 are configured to be inserted into receiving wells 110 of attachment structure 106. Although the use of tabs 112 is described, access cap 108 may include any other attachment mechanisms, such as by way of example only, magnets, receiving wells, threads, or adhesives, in order to removably attach the access cap 108 to the attachment section 106. In one example, the access cap 108 may be configured to provide a form-fit to attachment structure 106.

In one example, once coupled to the attachment structure 106, access cap 108 is secured to the attachment structure 106 by applying a twisting force to cause a rotating action of the access cap 108 with respect to the attachment structure 106, although other attachment mechanisms may be utilized. In one example, access cap 108 includes grip devices 114 on the top surface thereof to allow the medic to twist the access cap 108 into secured attachment with the attachment section 106, although the access cap 108 may include other elements in other locations on the access cap 108 to facilitate attachment. In one example, access cap 108 includes a gasket 116 located around an outer edge of the access cap to provide the occlusive seal when attached to the attachment structure 106, although the access cap may have other elements in other locations in order to provide the occlusive seal.

An exemplary operation of the wound dressing apparatus 100 of the present invention will now be described with reference to FIGS. 1A-1D. Patient interface 102 is attached to the skin of the patient extending around a wound on the patient's skin. For example, the wound dressing apparatus 100 may be applied to treat a chest wound, although other uses of the wound dressing apparatus may be contemplated. The patient interface 102 is attached in a manner that the access port 104 is located directly above the area of the wound with the access port 104 centrally located over the wound. The access port 104 provides the medic with access to the wound site if necessary.

Access cap 108 is attached to attachment section 106 to provide an occlusive seal for the wound dressing apparatus 100 over the wound site. The occlusive seal will cause pressure to build up in the wound site. The medic may remove the access cap 108 to release the built up pressure from the wound site. By way of example only, the access cap 108 may be removed by applying a twisting force to the access cap 108. The medic may then reattach the access cap 108 in order to reform an occlusive seal over the wound site. Alternatively, once the access cap 108 is removed, the medic may apply an attachable treatment device to the attachment section in order to apply the associated treatment as discussed below.

Referring now to FIGS. 2A-4B, exemplary treatment devices 200, 300, 400 capable of being operably attached to the wound dressing apparatus 100 after the access cap 108 is removed to provide an associated medical treatment are illustrated. Each of the treatment devices 200, 300, 400, includes one or more attachment mechanisms configured to mate the treatment device to attachment structure 106, such as, by way of example only, tabs 112. Tabs 112 are configured to be inserted into receiving wells 110 of attachment structure 106. In one example, once coupled to the attachment structure 106, each treatment device 200, 300, 400, is secured to the attachment structure 106 by applying a twisting force to cause a rotating action of the treatment device with respect to the attachment structure 106. In one example, each of the treatment devices 200, 300, 400 includes a gasket 116 located around an outer edge of the treatment device to provide the occlusive seal when attached to the attachment structure 106, although the access cap may have other elements in other locations in order to provide the occlusive seal.

Referring to FIGS. 2A-2D, an exemplary dual pump treatment device 200 which may be coupled to the wound dressing apparatus 100 (with access cap 108 removed) as illustrated in FIG. 1 is shown. The dual pump treatment device 200 includes receiving port 202, a first valve 204, a vent 206, and a second valve 208, although the dual pump treatment device could include other components and elements in other configurations. The dual pump device 200 provides a number of advantages including providing an effective treatment device that is utilized to provide wound suction in the field that requires minimal training. The dual pump device 200 also advantageously may be removably attached to the wound dressing apparatus 100 allowing for efficient cleaning and interchangeability with other treatment devices without the need to remove the adhesive attaching the wound dressing apparatus 100 to the skin of the patient.

Referring again to FIGS. 2A-2D, the receiving port 202 is configured to receive an airflow device capable of providing either a positive pressure or a negative pressure to chamber 210 in the dual pump device. By way of example only, receiving port 202 may receive a bulb pump, a syringe, or a billows bag, although other airflow devices may be contemplated.

The first valve 204 is located between the receiving port 202 and the wound site, which is covered by access port 104 when the dual pump device 200 is attached to the attachment section 106. The first valve 204, by way of example is a flapper valve configured to open and close the access port 104 in response to changes in pressure, although other types of valves that operate in other manners may be utilized.

Vent 206 is attached to the chamber 210. Vent 206 is configured to allow air to be released from the dual pump treatment device 200. A second valve 208 is located prior to the vent 206, although the second valve may be positioned in other locations of the dual pump device 200. The second valve 208 is a flapper valve configured to open and close vent 206 in response to changes in pressure, although other types of valves that operate in other manners may be utilized.

An exemplary operation of the dual pump device 200 when attached to the attachment section 106 of the wound dressing apparatus 100 will now be described with reference to FIGS. 2C-2D. The dual pump treatment device 200 is configured to provide manual suction to the wound area allowing for release of pressure caused by a tension pneumothorax, although other applications of the dual pump device 200 may be contemplated.

Patient interface 102 is attached to the skin of the patient extending around a wound or incision site on the patient's skin. The patient interface 102 is attached in a manner that the access port 104 is located directly above the area of the wound or incision site with the access port 104 centrally located over the wound or incision site. Access cap 108 is removed and the dual pump treatment device 200 is attached to the wound dressing apparatus 100.

The user inserts an airflow device capable of providing either a positive pressure or a negative pressure, such as a bulb pump, syringe, or billows bag, into the receiving port 202. The user operates the airflow device to alternately apply a positive or negative airflow pressure to chamber 210 of the dual pump treatment device 200 by, for example, manually squeezing a bulb pump or billows bag, or working the plunger back and forth on a syringe.

Referring to FIG. 2C, during a positive airflow from the airflow device air is pushed into chamber 210 through receiving port 202 forcing the first valve 204 to close over the access port 104, occluding the wound site. The positive airflow forces the second valve 208 to open allowing air to escape the dual pump treatment device 200 through vent 206.

Referring to FIG. 2D, during a negative air flow of pressure from the airflow device inserted into receiving port 202, air is pulled from the chamber 210 forcing the second valve 208 to close over vent 206. The negative airflow forces open the first valve 204, allowing air to escape the wound site and, by way of example only, relieving a tension pneumothorax. The user may alternately apply a positive and negative pressure by manually manipulating the attached airflow device to provide suction of the wound site without removing the airflow device from the receiving port 202.

Referring to FIGS. 3A-3B, an exemplary thoracic catheter device 300 which may be coupled to the wound dressing apparatus 100 (with access cap 108 removed) as illustrated in FIG. 1 is shown. The thoracic catheter device 300 includes a tubing 302, such as a Penrose tubing that provides access to a wound or incision site through the access port 104 for insertion of a chest tube when attached to wound dressing apparatus 100 as shown in FIG. 3B, although the thoracic catheter device 300 may provide other functions known in the art related to catheter devices.

An exemplary operation of the thoracic catheter device 300 will now be described with reference to FIG. 3B. Patient interface 102 is attached to the skin of the patient extending around a wound or incision site on the patient's skin. The patient interface 102 is attached in a manner that the access port 104 is located directly above the area of the wound or incision site with the access port 104 centrally located over the wound or incision site. Access cap 108 is removed and the thoracic catheter device 300 is attached to the wound dressing apparatus 100.

The medic feeds chest tube 304 through the tubing 302 and the access port 104 to insert the chest tube 302 into the wound or incision site. Once the chest tube is in place, the medic may optionally apply an adhesive, such as tape, to the tubing 302 and the chest tube 304 to secure the chest tube 304 to the thoracic catheter device 300 and to provide a complete occlusion over the wound site. The thoracic catheter device 300 is secured to the attachment section 106 to eliminate movement of the chest tube 304 after insertion. Chest tube 304 may optionally be connected to a manually operated pump device (not shown), such as a billows bag, syringe, or bulb pump, to provide blood and air removal from the wound site.

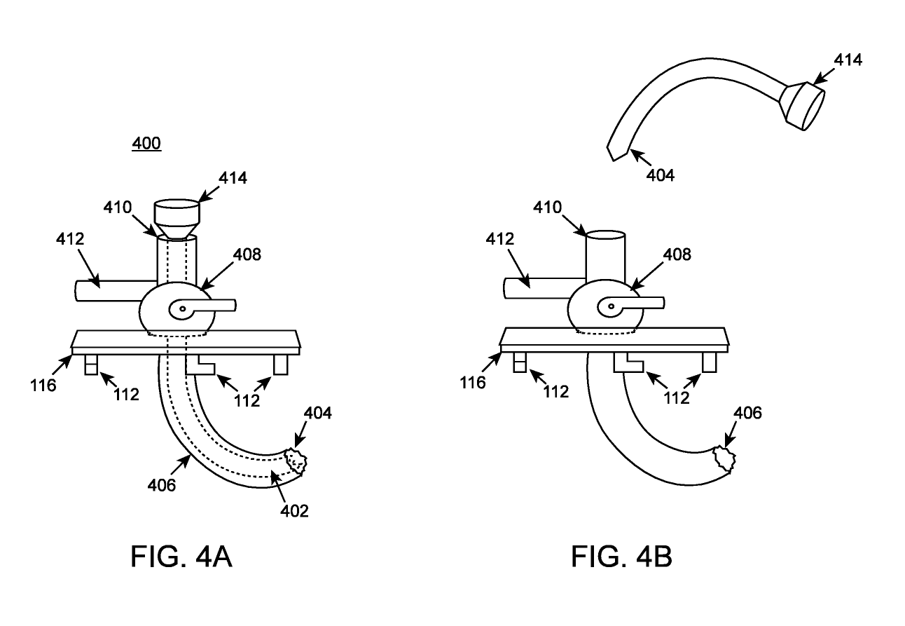

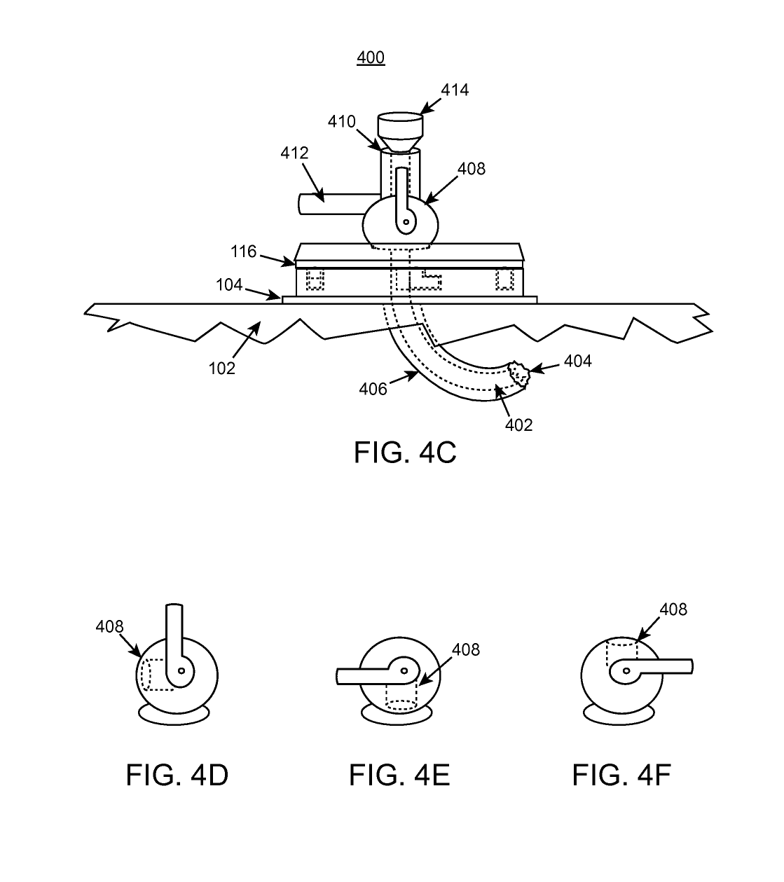

Referring to FIGS. 4A-4F, an exemplary thoracic trocar device 400 which may be coupled to the wound dressing apparatus 100 (with access cap 108 removed) as illustrated in FIG. 1 is shown. The thoracic trocar device 400 includes trocar 402, trocar tip 404, catheter 406, three-way stop cock 408, and receiving ports 410 and 412, although the thoracic trocar device 400 could include other elements and components in other configurations. The thoracic trocar device 400 advantageously allows insertion of the trocar quickly in the field without the need for multiple instruments, such as hemostats, which are typically used in such a treatment. The thoracic trocar device 400 further requires minimal training to insert and provides an effective, portable alternative to a chest tube. The thoracic trocar device 400 may be utilized to relieve pressure in the patient's chest caused by blood instead of air.

The trocar 402 includes trocar tip 404 which located at the end of the trocar 402 and is configured to provide insertion into the skin of the patient. The trocar 402 is coupled to the catheter 406. In one example, the catheter is three to four inches on one side, although other size catheters may be utilized. The thoracic trocar device 400 may be utilized with various diameters of tubing, for example 10 french to 32 french, depending on the treatment to be performed. By way of example, the device may be utilized for multiple breathing treatment protocols, such as chest tube or thoracic catheter emplacement, needle decompression of the chase, or as a cricothyrotomy tube, although other treatment protocols may be contemplated.

Three-way stop cock 408 is configured to be located above the access port 104 when the thoracic trocar device 400 is attached to the attachment section 106 of the wound dressing apparatus 100 (with the access cap 108 removed), although other valve device may be utilized. Referring now to FIGS. 4D-4F, the three-way stop cock includes three different positions to open and close receiving ports 410 and 412, as well as to remove the trocar 402. Referring again to FIGS. 4A-4C, receiving ports 410 and 412 are coupled to the three-way stop cock 408 and provide ventilation or attachment to various suction devices such as, by way of example only, an AMBU bag, syringe, bulb pump, or billows bag. Receiving port 412 may include removable cap 414 to close the receiving port 412.

An exemplary operation of the thoracic trocar device 400 will now be described with reference to FIG. 4C. Patient interface 102 is attached to the skin of the patient extending around a wound or incision site on the patient's skin. The patient interface 102 is attached in a manner that the access port 104 is located directly above the area of the wound or incision site with the access port 104 centrally located over the wound or incision site. Access cap 108 is removed and the thoracic trocar device 400 is attached to the wound dressing apparatus 100.

The trocar tip 404 is placed into the desired insertion point and forced through the opening created by the trocar 402. By way of example only, the medic may insert the trocar 402 by locating the patient's fifth rib at the mid-auxiliary line and pulling the skin tight prior to insertion. Once the catheter 406 is in place, the medic removes the trocar 402. The medic may then operatively adjust the three-way stop cock 408. Referring to FIGS. 4C-4E, the various positions of the three-way stop cock 408 are described.

As shown in FIG. 4D, the three-way stop cock 408 is in the full open position. The full open position allows the trocar 402 to be removed or emplaced. The full open position also allows blood to escape the thoracic trocar device 400 through receiving ports 410 and 412. In one example, the full open position may be utilized to provide ventilation of the patient during a cricothyrotomy by connecting a ventilation device to receiving port 410 and closing receiving port 412 using the medic's finger. As shown in FIG. 4E, the position of the three-way stop cock 408 opens receiving port 412. A suction device (not shown) such as a syringe, billows bag, or bulb pump may be inserted into receiving port 412 to provide suction. As shown in FIG. 4F, the three-way stop cock 408 is in the full closed position. The full closed position closes the access port 104 to provide occlusion to prevent blood or air from escaping through the thoracic trocar device 400. In one example, the full closed position is utilized immediately after emplacement and removal of the trocar 402.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Accordingly, the invention is limited only by the following claims and equivalents thereto.

What is claimed is:

1. A wound dressing apparatus comprising:
a patient interface configured to detachably attach to an area of skin on a patient and extend around a wound on the patient;
an access port extending from the patient interface, wherein the access port comprises an aperture in the patient interface configured to provide access to the wound;
an attachment structure extending about and out from an outer boundary of the access port; and
an access cap configured to be removably attached to the attachment structure to provide an occlusive seal over the wound when attached to the attachment structure.

2. The wound dressing apparatus of claim 1 wherein the patient interface further comprises an adhesive layer configured to detachably attach the patient interface to the area of skin on the patient.

3. The wound dressing apparatus of claim 1 wherein the access cap further comprises a gasket around an outer edge of the access cap to provide the occlusive seal when attached to the attachment structure.

4. The wound dressing apparatus of claim 1 wherein the attachment structure further comprises one or more attachment mechanisms configured to removably attach the access cap to the attachment structure.

5. The wound dressing apparatus of claim 1 further comprising a treatment device capable of being operably attached to the attachment structure after the access cap is removed.

6. The wound dressing apparatus of claim 5 wherein the treatment device comprises a pump device configured to provide suction of an area around the wound.

7. The wound dressing apparatus of claim 6 wherein the pump device comprises:
a receiving port configured to receive an airflow device capable of providing either a positive pressure or a negative pressure to a chamber in the pump device;
a first valve located between the receiving port and the wound wherein the first valve is configured to occlude the wound in response to a positive pressure and to open in response to a negative pressure;
a vent attached to the chamber; and
a second valve located over vent wherein the second valve is configured to close in response to a negative pressure and open in response to a positive pressure.

8. The wound dressing apparatus of claim 7 wherein the airflow device comprises a bulb pump, a syringe, or a billows bag.

9. The wound dressing of claim 5 wherein the treatment device comprises a thoracic catheter.

10. The wound dressing of claim 5 wherein the treatment device comprises a thoracic trocar.

11. A method of treating a wound on a patient comprising:
providing a wound dressing apparatus comprising a detachable patient interface configured to detachably attach to an area of skin on the patient and extend around the wound on the patient, an access port extending from the patient interface, wherein the access port comprises an aperture in the wound dressing apparatus configured to provide access to the wound, an attachment structure extending about and out from an outer boundary of the access port, and an access cap configured to be removably attached to the attachment structure to provide an occlusive seal over the wound when attached to the attachment structure;
attaching the detachable patient interface to the area of skin on the patient extending around the wound of the patient; and
removing the access cap from the attachment structure to relieve pressure from the wound.

12. The method of claim 11 wherein the patient interface comprises an adhesive layer configured to attach the patient interface to the area of skin on the patient.

13. The method of claim 11 wherein the access cap comprises a gasket around an outer edge of the access cap to provide the occlusive seal when attached to the attachment structure.

14. The method of claim 11 wherein the attachment structure further comprises one or more attachment mechanisms configured to removably attach the access cap to the attachment structure.

15. The method of claim 11 further comprising:
providing a treatment device capable of being operably attached to the attachment structure after the access cap is removed; and
attaching the treatment device to the attachment structure of the wound dressing apparatus.

16. The method of 15 wherein the treatment device comprises a pump device configured to provide suction of an area around the wound.

17. The method of claim 16 wherein the pump device comprises a receiving port configured to receive an airflow device capable of providing either a positive pressure or a negative pressure to a chamber in the pump device, a first valve located between the receiving port and the wound wherein the first valve is configured to occlude the wound in response to a positive pressure and to open in response to a negative pressure, a vent attached to the chamber, and a second valve located over the vent wherein the second valve is configured to close in response to a negative pressure and open in response to a positive pressure, the method further comprising:
inserting the airflow into the receiving port; and
operating the airflow device to provide an alternating positive and negative pressure in the chamber.

18. The method of claim 17 wherein the airflow device comprises a bulb pump, a syringe, or a billows bag.

19. The method of claim 15 wherein the treatment device comprises a thoracic catheter.

20. The method of claim 15 wherein the treatment device comprises a thoracic trocar.
